(19) United States
(12) Patent Application Publication
(10) Pub. No.: US 2024/0033349 A1
(43) Pub. Date: Feb. 1, 2024

(54) VACCINE AND METHOD OF PROTECTION AGAINST CORONAVIRUS INFECTION

(71) Applicant: Musa Tazhudinovich ABIDOV, Moscow (RU)

(72) Inventors: Musa Tazhudinovich ABIDOV, Moscow (RU); Avni AKVARDARf, Moscow (RU)

(21) Appl. No.: 18/016,652

(22) PCT Filed: Jun. 29, 2021
(86) PCT No.: PCT/RU2021/000278

§ 371 Date: Jan. 17, 2023

(30) Foreign Application Priority Data
Jul. 17, 2020 (RU) 2020123856

(51) Int. Cl. A61K 39/39; A61K 31/502; A61P 31/14

(52) U.S. Cl. CPC A61K 39/39; A61K 31/502; A61P 31/14; A61K 2039/55511

(57) ABSTRACT

The invention is related to medical and biological applications and is intended to prevent and treat coronavirus infections by applying phthalhydrazide derivatives, including Tamerit, with immunomodulatory activity independently or in combination with antiviral drugs of different chemical structure.

A method for the prevention of coronavirus infection is presented, characterized by the fact that to increase the clinical and laboratory efficacy achieved by antiviral agents of the azoloazine series (Triazavirin®, Maktavirin®), antimalarials and preparations of interferon, in combination with the above preparations a preparation of aminophthalhydrazide derivatives salt, in the form of dihydrate, monohydrate, anhydrate, in any crystalline form, including Tamerit, in a dose from 0.01 to 4000 mg/kg to a subject in need is used. This pattern of using the drug, (according to the results of preclinical studies) showed that Tamerit provides an aggregate level of protection to 100%, exceeding the level of protection using only antiviral drugs by 30-50% and by 1.5-2 weeks reduces the duration of the acute course and the disease as a whole.

### THE FIELD OF TECHNOLOGY COVERED BY THE INVENTION

The invention is related to medical and biological applications and is intended to prevent and treat coronavirus infections by applying phthalhydrazide derivatives, including Tamerit, with immunomodulatory activity independently or in combination with antiviral drugs of different chemical structure.

### THE STATE OF THE ART

The beginning of the 21st century was marked by the emergence of two new coronaviruses (CoV) with remarkably high virulence and pathogenicity for this group of viruses, and the lesions they cause are often fatal [1-5]. Severe Acute Respiratory Syndrome Coronavirus (SARS-CoV) was first diagnosed in 2003 [6-9]. In the early days of SARS, some concern was expressed regarding its emergence and spread, however, the situation has changed dramatically and virtually disappeared with the lack of new cases and, as a result, funding for research in this area has stopped. A decade later, however, a second new coronavirus, the Middle East respiratory syndrome pathogen (MERS-CoV), first identified in Saudi Arabia, appeared [3, 10]. Since it was first detected, SARS-CoV has infected about 8,000 people, and lethal outcomes have been reported in about 10% of infected patients [11]. At the same time, it should be noted that although MERS-CoV is not as prevalent as SARS-CoV, it is more pathogenic and virulent and causes a higher percentage of deaths, equal to 35-50% of all diagnosed cases of infection [3-5, 9, 12-15].

Recently, other CoVs have also been detected in animal populations, which not only increases the likelihood of similar outbreaks recurring in the near future, but also the occurrence of outbreaks caused by fundamentally new CoVs [11,16-20]. Both SARS-CoV and MERS-CoV zoonotic viruses are capable of causing much more severe illnesses than those typically associated with CoV, making them potential biological warfare agents (BPAs) and causing lesions a global problem for modern public health as well as military medicine as applied to the military. SARS-CoV and MERS-CoV infection leads to severe lung injury such as acute lung injury (ALI) followed by pulmonary edema and respiratory failure in the absence of cardiovascular pathology. In some cases, the lesions described above continue and develop dynamically into a more severe form of ALS—acute respiratory distress syndrome (ARDS) [21-23]. In order to survive under the conditions described above, the immune system must not only block the reproduction of pathogenic viruses in the body through specific mechanisms but also actively fight the damage at the level of, first of all, the respiratory system, as well as level out the damage from the immune system [24-26].

The development of the above syndromes also occurs in the new coronavirus infection caused by SARS-CoV-2. When examining patients with the above-mentioned infection, immune dysfunction was found to play a key role in its pathogenesis [27,28,29,30]. At the same time, the presence of lymphopenia and the absence of neutropenia are noted. Peripheral blood lymphocytes are predominantly cells with HLA-DR and CD38 markers. They are related to subpopulations of perform and/or granulin-positive CD8⁺ T-lymphocytes or inflammatory Th17− cells. Considering that the latter produce pronounced tissue damaging effects, as well as immunosuppressive activity, the consequence of such changes was significant lung tissue damage in the form of diffuse alveolar damage, and the development of ARDS. Such changes are consistent with the high expression of the SARS-CoV-2 receptor on pneumocytes. Recent studies show that one of the biologically plausible mechanisms of lung damage in SARS-CoV-2 is antibody-dependent amplification (ADA) [29,30]. This case has also been reported in infections with dengue, Zika, Ebola and human immunodeficiency viruses. Antibody-dependent amplification is caused not only by neutralizing antibodies, but also by non-neutralizing antibodies. For SARS-CoV-2, ASU is provided by cells with the CD32 phenotype, which include monocytes and macrophages and alveolar macrophages.

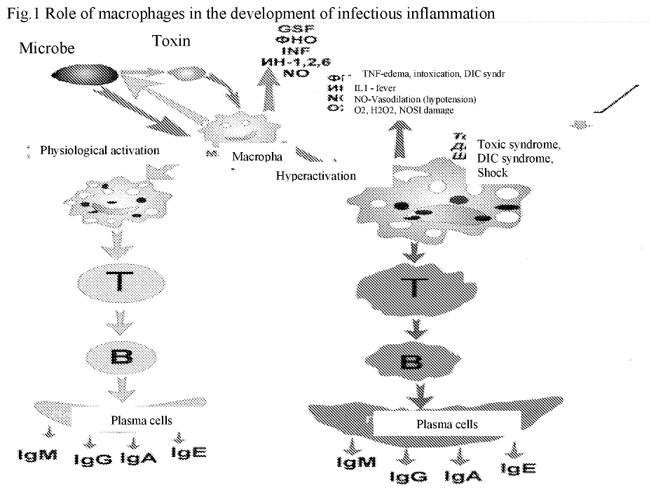

Therefore, in infections caused by coronaviruses, there is a powerful inflammatory response that develops against the background of immune system imbalance and is mediated by a powerful “cytokine explosion” induced by increased release of proinflammatory cytokines. In this regard, it is necessary to search for means that can balance these changes. In this case, the focus should be primarily on anti-inflammatory agents (FIG. 1).

Abidov M. T. (1980-1994) first discovered some of the most common and obligatory connections in the development of inflammatory reactions during the study of the pathogenesis of various etiologies of infectious inflammatory diseases. Macrophages are the first cellular elements that encounter the microorganism after they penetrate the epithelium and skin. Their further “behavior” is what determines the severity and nature of the body's response to the introduction of the pathogen. Excessive macrophage activity leads to the release of large amounts of proinflammatory cytokines such as, tumor necrosis factor, interleukins, nitro compounds, prostaglandins and reactive oxygen radicals. It is these substances that produce para- and endocrine effects, causing local and general inflammatory reactions. Involvement in the pathological process of monocytic/macrophage cells, followed by their activation and production of a variety of biologically active compounds that aggravate cellular and general disorders, is one of the most universal links in the development of inflammation. Therefore, a logical strategy in the pathogenetic therapy of acute inflammatory diseases, regardless of the etiological factor, seems to be the impact on the key link in order to inhibit (reversibly) the excessive activity of monocytes/macrophages. Later it was confirmed (M. Abidov's Doctor of Medicine thesis 1993) that the acute period of inflammation always corresponds to a distorted hyperactive response of macrophages. This confirmed the theory that hyperactivated macrophages should be inhibited during acute inflammation. Whereas many researchers have considered inflammation to be a decrease in the function of the immune system. Abidov M.'s theory was later confirmed by Volk (1999) “Macrophage deactivation is the most important strategy in the sepsis treatment”.

Afterward, it was confirmed in the experiment and in the clinical practice that by regulating the function of these cells, it is possible to treat a wide range of diseases.

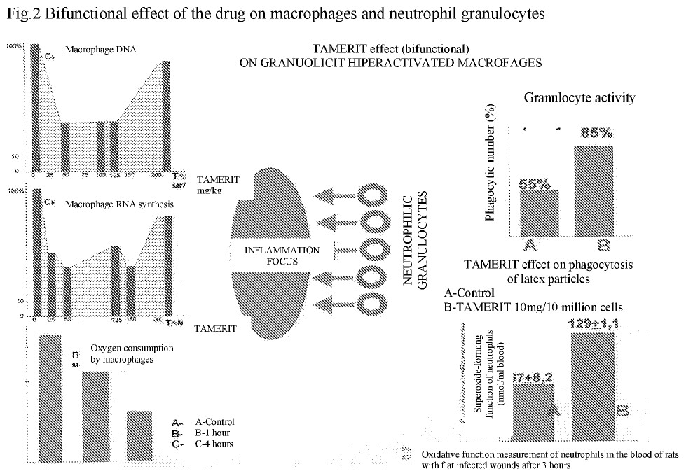

It was also found that along with inhibition of macrophage hyperactivity, Tamerit simultaneously activated the neutrophil microbicidal system and thereby strengthened the body's resistance to microbial invasion (FIG. 2). These prerequisites provided an opportunity to create the drug Tamerit, one of the derivates of phthalhydrazide with anti-infective features with a possible dosage for use in medicine and veterinary medicine from 0.01 mg/kg to 4000 mg/kg.

Bifunctional effect of Tamerit was discovered in, firstly, when the drug reduces the hyperactivity of macrophages with a subsequent decrease in pro-inflammatory cytokines and reactive oxygen radicals, and secondly, it activates phagocytic and microbicidal activity of neutrophil granulocytes located near the focus of inflammation.

The use of a macrophage function modulator represents a new strategy in the treatment of many inflammatory diseases independent of the etiological factor.

The drug formula is aminodihydrophthalazindione sodium (5-amino-1,2,3,4-tetrahydrophthalazine-1,4-dione sodium salt); a derivative of synthetic phthalhydrazide derivatives. The other derivatives of phthalhydrazide with a similar formula, as well as other salts of the compound, may have therapeutic properties (to a greater or lesser extent). Since the structure of a substance is what determines its properties. Hence, substances with similar structures have similar properties, differing in some details. Thus, it is reasonable to generalize, until proven otherwise, that all derivatives of phthalhydrazide would have similar therapeutic properties, although in varying degrees of intensity.

The key mechanisms of the immunotropic action of “Tamerit”:
At the level of the system of non-specific immunological resistance—Tamerit suppresses (reversibly, for 8-12 hours) hyperactivity of macrophages, reduces the production of reactive oxygen species and other acute phase proteins involved in the development of toxic syndrome; normalizes the functional state of macrophages, restores the regulatory function, increasing the antibacterial activity of neutrophil granulocytes, increases phagocytosis and increases the non-specific defense of the body;
at the level of the cytokine system—Tamerit reduces the excess synthesis of TNF, interleukin-1; at the level of T- and B-immunological activity systems there are no effects;
anti-infective activity: effectiveness of the drug is shown for herpes infection, hemorrhagic fevers and other infectious diseases occurring with toxic and septic syndrome. (Abidov M. T. Toxic syndrome, pathogenesis, correction methods 1994)

Considering the danger of coronavirus infections to humans, including SARS-CoV-2, the search for effective means of prevention and therapy of these infections seems significant, and considering the imbalance in the immune system caused by each of the infectious agents in the body, the priority in this plan may be given to drugs with a preferential immunotropic effect.

However, no information on studies of derivatives of synthetic phthalhydrazide (Tamerit) in the prevention and treatment of coronavirus infection at the technical level was found. These studies have been conducted for several years. On the one hand, Tamerit has indeed been used in the treatment of a number of virus infections, but on the other hand, since the effect of each virus on the body has a number of specific characteristics, it is incorrect to extrapolate the data obtained on one virus infection to another. There is no reason to speculate that the data on the treatment of hepatitis C can be extrapolated to the treatment of coronavirus infection, since these diseases not only have different localizations, but also the viruses that cause them have tropism to different cellular receptors and, accordingly, develop inside different cells. And since derivatives of synthetic phthalhydrazide derivatives affect cells of the body rather than the virus, no prediction of how they will affect the development of diseases with tropism to different cells is possible. Moreover, there is no information reported at the technical level that phthalhydrazide is suitable for the prevention of any RNA viral diseases. Thus, the use of phthalhydrazide and its derivatives, such as Galavit (author and developer Abidov M. T. 1994) having a similar formula is different from Tamerit by structural and pharmacological properties (Rybakov et al.), Tameron (developer Abidov M. T. Pigulevsky A. V.), a crystalline, lyophilized powder, for protection against coronavirus infection is not only new but also unobvious to the average specialist at the level of technology.

However, we have found that Tamerit demonstrated higher therapeutic efficacy and safety in the treatment of various diseases. Clinical examples confirmed that Tamerit, unlike other derivatives, has no toxic, cumulative, mutagenic or teratogenic effects. We also found that failure to observe certain parameters during production, all derivatives of phthalhydrazide form non-safe metabolites and conjugates, when introduced into the body in any way, respectively, produce undesirable side effects. For example, no salt of Luminol (3-Aminophthalhydrazide) is used in practical medicine due to extensive side and unwanted effects, unlike Tamerit, Tameron, or Galavit.

As studies have shown, the use of Tamerit can be justified both in combination with antiviral medications and as part of a physical mixture with the mentioned drugs. The latter are generally represented by physical mixtures in which each drug (one or more) and excipients are contained in a specified weight ratio.

The new approach we propose will allow the formation of immunity to virus infection regardless of its etiological nature or the sensitivity of the causative strain to antiviral medicines.

The method of prevention and treatment of viral infections using combinations of chemo preparations with a synergistic mechanism of action is also known as “A method of combining nucleosides to achieve synergism of their antiviral effect”. [31], “A method of combining zidovudine with a nucleoside to achieve synergism of their antiviral effect” [32], “A method of combining nucleosides to achieve synergism of their antiviral effect” [33], “A method of combining interferon and a heterocyclic compound to achieve a synergistic effect of the drugs against viruses” [34], “A study of the combined effect of remantadine and ribavirin on experimental flu infection” [35], “A study of the combined effect of remantadine and ribavirin on the reproduction of Sindbis virus in cell culture”. [36], “Combined use of antiviral drugs” [37]. The method allows increasing the body's resistance to viral infections.

The invention is aimed at solving problems: increasing the efficiency of the method, expanding the indications for its use, and expanding the range of antiviral agents. These objectives are achieved through the combined use of an immunomodulatory agent with antiviral chemopreparations, regardless of the nature of the mentioned pharmacological agents and methods of production. Contraindications: not identified.

### THE ESSENCE OF THE INVENTION

Since protection against coronavirus infection is an objectively evident phenomenon and is a property of derivatives of synthetic phthalhydrazide derivatives (Tamerit), and according to Rospatent Order N 236 of 27 Dec. 2018 “On approval of the Guidelines for administrative procedures and actions in the provision of public service for state registration of inventions and granting patents for inventions and their duplicates”:

Technical results include the results representing the phenomenon, feature, as well as the technical effect resulting from the phenomenon, feature, objectively manifested in the implementation of the method or in the manufacture or use of the product, including the use of the product obtained directly by the method embodying the invention and, as a rule, characterized by physical, chemical or biological parameters, then

the technical result of this invention is to expand the indications for the use of the drug Tamerit (derivative of phthalhydrazide)—with immunocorrective and anti-inflammatory properties, which has an antiviral effect against coronavirus infections with independent use or in combination with antiviral drugs.

Tamerit can be a pharmaceutical platform for the creation of highly effective anti-infective drugs for emergency prevention and treatment of coronavirus infections, and not only.

As mentioned above, Tamerit, as well as phthalhydrazides of other chemical structure, such as Galavit, Tameron are not described in the literature as a measure of emergency prevention and therapy of coronavirus infections.

### INFORMATION CONFIRMING THE ESSENCE OF THE INVENTION

The method is designed for the simultaneous prevention, therapy and rehabilitation phase of treatment of the organism (protection) infected with coronaviruses. The vaccine is intended to protect against coronavirus infection as an adjuvant and contains Tamerit in effective amounts as well as pharmaceutically acceptable carriers and/or diluents from those traditionally used.

The examples confirm the effectiveness of Tamerit in the prevention and therapy of coronavirus infections.

### EXAMPLE 1

### Antiviral Activity of Tamerit in an Experimental Model of SARS Infection

In a series of experiments on male purebred white mice weighing 16-18 g received from “Stolbovaya” nursery, SARS model (strain SARS-Covktp3) or the effectiveness of Tamerit was studied. The drug was administered parenterally (subcutaneously) according to the standard scheme (24 h before infection, 24 h, 48 h, 72 h, 96 h, and 120 h after infection). The animals were infected with the SARS-Cov virus at a dose of 10 LD₅₀. The animals were monitored for 14 days, recording the number of live and dead animals daily. The results are shown in Table 1 and 2.

TABLE 1
Protective efficacy of Tamerit in a model of experimental
coronavirus infection caused by SARS
(SARS-Covktp3)

| | | | Number | | |
|---|---|---|---|---|---|
| | | | of | | |
| | | Contagious | animals | | |
| | | dose of the | in the | Survival | |
| | Dose, | pathogen, | group, | rate, | Protection, |
| Drug | mg/ml | LD₅₀ | heads | % | % |
| Tamerit | 0.05 | 10 | 10 | 20 (2 ÷ 56) | 20 (2 ÷ 56) |
| Tamerit | 0.1 | 10 | 10 | 20 (2 ÷ 56) | 20 (2 ÷ 56) |
| Tamerit | 0.15 | 10 | 10 | 40 (12 ÷ 74) | 40 (12 ÷ 74) |
| Tamerit | 0.2 | 10 | 10 | 40 (12 ÷ 74) | 40 (12 ÷ 74) |
| Tamerit | 0.25 | 10 | 10 | 50 (19 ÷ 81) | 50 (19 ÷ 81) |
| Control | — | 10 | 10 | 0 (0 ÷ 31) | — |

As the results presented in Tables 1 and 2 show Tamerit under the same conditions protected infected animals from SARS or MERS virus infection at the level of 20% to 50% against 100% lethality in the controls. The used dose changing of the drug had no noticeable positive effect, although we must admit that using Tamerit in doses of 200-250 mcg/ml, it allowed to obtain a protection effect of 50%.

TABLE 2
Protective efficacy of Tamerit on a model of coronavirus
infection caused by MERS-CovN3/Jordanp3 virus.

| | | | Number | | |
|---|---|---|---|---|---|
| | | | of | | |
| | | Contagious | animals | | |
| | | dose of the | in the | Survival | |
| | Dose, | pathogen, | group, | rate, | Protection, |
| Drug | mg/ml | LD₅₀ | heads | % | % |
| Tamerit | 0.05 | 10 | 10 | 20 (2 ÷ 56) | 20 (2 ÷ 56) |
| Tamerit | 0.1 | 10 | 10 | 40 (12 ÷ 74) | 40 (12 ÷ 74) |
| Tamerit | 0.15 | 10 | 10 | 40 (12 ÷ 74) | 40 (12 ÷ 74) |
| Tamerit | 0.2 | 10 | 10 | 50 (19 ÷ 81) | 50 (19 ÷ 81) |
| Tamerit | 0.25 | 10 | 10 | 50 (19 ÷ 81) | 50 (19 ÷ 81) |
| Control | — | 10 | 10 | 0 (0 ÷ 31) | — |

The results showed some activity of Tamerit against coronaviruses, the causative agents of SARS and MERS, although it should not be overlooked that Tamerit has a certain activity against coronaviruses:
this effect is nonspecific and seems to be caused by the immunotropic effects of Tamerit, in particular its anti-inflammatory effect;
the level of protection against the use of the drug was maximum 50% against 100% lethality in the control. However, considering the lack of reliable differences compared to the control, it should be regarded as a tendency to increase the body's resistance to the coronavirus infection pathogens used in the study;
it seems that the most objective place of Tamerit in the anti-infective protection against coronaviruses is its combined use with antiviral agents, mainly directed, for example, against RNA viruses.

### EXAMPLE 2

### The Combination of Tamerit with Triazavirin and Maktavirin with Tamerit in an Experimental Model of SARS Infection

The efficacy of triazavirin in combination with Tamerit and maktavirin in combination with Tamerit was studied in a series of experiments on male purebred white mice weighing 16-18 g received from Stolbovaya nursery on the SARS model (strain SARS-Covktp3). The antiviral agents were administered orally and Tamerit parenterally (subcutaneously) according to the same schemes (24 h before infection, 24 h, 48 h, 72 h, 96 h and 120 h after infection) to SARS-Cov infected people at a dose of 10 LD₅₀. The animals were monitored for 14 days, recording the number of live and dead animals daily. The results are shown in Table 3.

The data show that the antiviral drugs triazavirin and maktavir ensured the immunity to SARS-CoV infection among infected animals, depending on the dose used, at the level of 20% to 70%, with the most effective doses of the antiviral drugs used being 10.0 mg/ml, 100.0 mg/ml and 1000.0 mg/ml.

TABLE 3
A comparative evaluation of the protective efficacy of triazavirin
in combination with Tamerit and maktavirin in combination
with Tamerit in an experimental model of SARS
(SARS-Covktp3)

| | | Conta- | | | |
|---|---|---|---|---|---|
| | | gious | Number | | |
| | | dose | of | | |
| | | of the | animals | | |
| | | path- | in the | | |
| | Dose, | ogen, | group, | Survival rate, | Protection, |
| Drug | mg/ml | LD₅₀ | heads | % | % |
| Triazavirin + | 0.01 | 10 | 10 | 20 (2 ÷ 56) | 20 (2 ÷ 56) |
| Tamerit | 0.05 | | | | |
| Triazavirin + | 1.0 | 10 | 10 | 50 (19 ÷ 81) | 50 (19 ÷ 81) |
| Tamerit | 0.1 | | | | |
| Triazavirin + | 10.0 | 10 | 10 | 70 (35 ÷ 93)* | 70 (35 ÷ 93) |
| Tamerit | 0.15 | | | | |
| Triazavirin + | 100.0 | 10 | 10 | 90 (56 ÷ 100)* | 90 (56 ÷ 100) |
| Tamerit | 0.2 | | | | |
| Triazavirin + | 1000.0 | 10 | 10 | 100 (69 ÷ 100)* | 100 (69 ÷ 100) |
| Tamerit | 0.25 | | | | |
| Maktavirin + | 0.01 | 10 | 10 | 20 (2 ÷ 56) | 20 (2 ÷ 56) |
| Tamerit | 0.05 | | | | |
| Maktavirin + | 1.0 | 10 | 10 | 40 (12 ÷ 74) | 40 (12 ÷ 74) |
| Tamerit | 0.1 | | | | |
| Maktavirin + | 10.0 | 10 | 10 | 70 (35 ÷ 93)* | 70 (35 ÷ 93) |
| Tamerit | 0.15 | | | | |
| Maktavirin + | 100.0 | 10 | 10 | 90 (56 ÷ 100)* | 90 (56 ÷ 100) |
| Tamerit | 0.2 | | | | |
| Maktavirin + | 1000.0 | 10 | 10 | 100 (69 ÷ 100)* | 100 (69 ÷ 100) |
| Tamerit | 0.25 | | | | |
| Tamerit | 0.05 | 10 | 10 | 20 (2 ÷ 56) | 20 (2 ÷ 56) |
| Tamerit | 0.1 | 10 | 10 | 20 (2 ÷ 56) | 20 (2 ÷ 56) |
| Tamerit | 0.15 | 10 | 10 | 40 (12 ÷ 74) | 40 (12 ÷ 74) |
| Tamerit | 0.2 | 10 | 10 | 40 (12 ÷ 74) | 40 (12 ÷ 74) |
| Tamerit | 0.25 | 10 | 10 | 50 (19 ÷ 81) | 50 (19 ÷ 81) |
| Control | — | 10 | 10 | 0 (0 ÷ 31) | — |

*Differences with control are significant at p < 0.05.

Tamerit under the same conditions protected infected animals from SARS virus infection at the level of 20% to 50% against 100% lethality in the control. However, the combination of triazavirin or maktavirin with Tamerit significantly increased the level of antiviral protection and, depending on the used dose of each drug in combination, increased by 30-50% compared to the use of Tamerit alone and by 30-40% depending on the use of each of the antiviral drugs. Thus, the clearly expressed dose-dependent ability of Tamerit to increase anti-infective activity when used in combination with conventional antiviral treatments against SARS-CoV RNA virus with 100% survival rate and 100% protection against infection was revealed.

### EXAMPLE 3

### The Antiviral Activity of the Combination of Triazavirin with Tamerit and Maktavirin with Tamerit in an Experimental MERS Infection Model

In a series of experiments, the efficacy of triazavirin in combination with Tamerit and maktavirin in combination with Tamerit was studied in a MERS model (strain MERS-CovN3/Jordanp3) in 16-18 g male purebred mice obtained from the cattery. The antiviral agents were administered orally, and Tamerit—parenterally (subcutaneously) according to the same schemes (24 h before infection, 24 h, 48 h, 72 h, 96 h and 120 h after infection) to MERS-CoV-infected patients at a dose of 10 LD₅₀. The animals were monitored for 14 days, recording the number of live and dead animals daily. The results are shown in Table 4.

As the data in Table 4 show, in the MERS model the efficacy of the combined use of antiviral drugs with Tamerit was significantly higher than that registered in the case of each of them separately. At the same time, a clearly expressed dose-dependent ability of Tamerit to increase the anti-infective activity when used in combination with traditional antiviral agents against the MERS-CovN3 RNA virus with 100% survival rate and 100% protection against infection was detected.

TABLE 4
A comparative evaluation of the protective efficacy of triazavirin in combination with
Tamerit and maktavirin in combination with Tamerit in an experimental
MERS model (MERS-CovN3/Jordanp3 virus)

| | | Contagious | Number of | | |
|---|---|---|---|---|---|
| | | dose of the | animals in | Survival | |
| | Dose, | pathogen, | the group, | rate, | Protection, |
| Drug | mg/ml | LD₅₀ | heads | % | % |
| 1 | 2 | 3 | 4 | 5 | 6 |
| Triazavirin + | 0.01 | 10 | 10 | 10 (0 ÷ 44) | 10 (0 ÷ 44) |
| Tamerit | 0.05 | | | | |
| Triazavirin + | 1.0 | 10 | 10 | 60 (26 ÷ 83) | 60 (26 ÷ 83) |
| Tamerit | 0.1 | | | | |
| Triazavirin + | 10.0 | 10 | 10 | 80 (44 ÷ 98)* | 80 (44 ÷ 98) |
| Tamerit | 0.15 | | | | |
| Triazavirin + | 100.0 | 10 | 10 | 100 (69 ÷ 100)* | 100 (69 ÷ 100) |
| Tamerit | 0.2 | | | | |
| Triazavirin + | 1000.0 | 10 | 10 | 100 (69 ÷ 100)* | 100 (69 ÷ 100) |
| Tamerit | 0.25 | | | | |
| Maktavirin + | 0.01 | 10 | 10 | 20 (2 ÷ 56) | 20 (2 ÷ 56) |
| Tamerit | 0.05 | | | | |
| Maktavirin + | 1.0 | 10 | 10 | 40 (12 ÷ 74) | 40 (12 ÷ 74) |
| Tamerit | 0.1 | | | | |
| Maktavirin + | 10.0 | 10 | 10 | 70 (35 ÷ 93)* | 70 (35 ÷ 93) |
| Tamerit | 0.15 | | | | |
| Maktavirin + | 100.0 | 10 | 10 | 90 (56 ÷ 100)* | 90 (56 ÷ 100) |
| Tamerit | 0.2 | | | | |
| Maktavirin + | 1000.0 | 10 | 10 | 100 (69 ÷ 100)* | 100 (69 ÷ 100) |
| Tamerit | 0.25 | | | | |
| Triazavirin | 0.01 | 10 | 10 | 20 (2 ÷ 56) | 20 (2 ÷ 56) |
| Triazavirin | 1,0, | 10 | 10 | 20 (2 ÷ 56) | 20 (2 ÷ 56) |
| Triazavirin | 10.0 | 10 | 10 | 40 (12 ÷ 74) | 40 (12 ÷ 74) |
| Triazavirin | 100.0 | 10 | 10 | 50 (19 ÷ 81) | 50 (19 ÷ 81) |
| Triazavirin | 1000.0 | 10 | 10 | 50 (19 ÷ 81) | 50 (19 ÷ 81) |
| Maktavirin | 0.01 | 10 | 10 | 10 (0 ÷ 44) | 10 (0 ÷ 44) |
| Maktavirin | 1,0, | 10 | 10 | 20 (2 ÷ 56) | 20 (2 ÷ 56) |
| Maktavirin | 10.0 | 10 | 10 | 40 (12 ÷ 74) | 40 (12 ÷ 74) |
| Maktavirin | 100.0 | 10 | 10 | 50 (19 ÷ 81) | 50 (19 ÷ 81) |
| Maktavirin | 1000.0 | 10 | 10 | 50 (19 ÷ 81) | 50 (19 ÷ 81) |
| Tamerit | 0.05 | 10 | 10 | 20 (2 ÷ 56) | 20 (2 ÷ 56) |
| Tamerit | 0.1 | 10 | 10 | 40 (12 ÷ 74) | 40 (12 ÷ 74) |
| Tamerit | 0.15 | 10 | 10 | 40 (12 ÷ 74) | 40 (12 ÷ 74) |
| Tamerit | 0.2 | 10 | 10 | 50 (19 ÷ 81) | 40 (19 ÷ 81) |
| Tamerit | 0.25 | 10 | 10 | 50 (19 ÷ 81) | 50 (19 ÷ 81) |
| Control | — | 10 | 10 | 0 (0 ÷ 31) | — |

*Differences with control are significant at p < 0.05.

### EXAMPLE 4

### The Efficacy of Tamerit in the Complex Therapy of Patients with New Coronavirus Infection COVID-19

The disease clinical picture is extremely polymorphic, in our practical experience we can distinguish at least four main variants of the infection depending on the predominant localization of the lesion:
Influenza-like version—affects mainly the upper respiratory tract, accompanied by fever, intestinal discomfort and diarrhea, loss of sense of smell and taste, cough (mostly dry), shortness of breath, difficulty in breathing, in most cases (clinically confirmed) quickly progresses to primary viral pneumonia;
infiltrative version has similar features to the course of infiltrative form of tuberculosis infection: with massive lymphocytic-neutrophil infiltration of the lungs, accumulation in the focus of reactive, oxygenated anion-radicals, leading ultimately to destructive changes in lung tissue.
toxic version—clinical symptoms are caused by progressive “cytokine storm”, microcirculatory disorders, disruption of the cell membrane triggering arachidonic cascade, synthesis of group E and F2a prostaglandins, prostacyclin and thromboxane imbalance, blood coagulation disorders, development of DIC syndrome, development of respiratory distress syndrome, pulmonary edema increase, brain edema further toxic shock, toxic-septic shock . . .
complicated version—caused by hyperergic immune system response, characterized by massive damage of lung parenchyma, formation of fibrosis foci, widespread vasculitis and, consequently, vascular disorders in different organs, including the brain. This version involves prolonged treatment and rehabilitation of patients with coronavirus infection until the clinical symptoms of the disease completely disappear in order to prevent disability.

The clinical picture of a new coronavirus infection has multiple forms, but the clinical course of the infection in the patients we observed has confirmed our assumptions. As a result, a new pathogenetic approach to the prevention and treatment of infection in the phase of acute clinical manifestations and at the rehabilitation stage was developed.

Since the beginning of the epidemic period, 26 people with coronavirus infection have been under our observation:

Group 1-5 patients (family), were in direct contact with an infected patient; patients had minimal clinical manifestations—sore throat (100%), perspiration (100%), dry cough (100%), muscle and joint tenderness (80%), disorders of smell and taste (60%). The contact with patients with new coronavirus infection COVID-19 was confirmed by small blood alterations (leukopenia, neutropenia, lymphocytosis). A CT scan of the chest organs did not reveal any pathology in the patients (100%). In all patients of this group, Tamerit injections of 200 mg 3 times a day, intramuscularly, for 3-4 days were administered to prevent the disease. After 3-4 days, patients had complete regression of symptoms. For prophylactic purposes, treatment with Tamerit 100 mg intramuscularly once a day was continued for another 3 weeks. All patients were treated as outpatients and isolated. No one of them got infected with the new coronavirus infection COVID-19 after contact with the patients.

Tamerit in this group of patients was used as monotherapy as a drug with immunomodulatory (inducer of endogenous interferon), anti-inflammatory properties (inhibitor of proinflammatory cytokine synthesis) and having its own antiviral effect against coronavirus infections.

Group 2-6 patients, patients in this group had all symptoms of new coronavirus infection in varying degrees of severity: sore throat (100%), perspiration (100%), dry cough (84%), muscle and joint pain (84%), chest tightness and shortness of breath (100%), olfactory and taste disorders (100%), temperature 37-38° C. (100%), headache (100%), hemoptysis in one patient (16%), in blood tests—leukopenia, neutropenia, lymphocytosis (84%). Positive test for COVID-19 in 100% of patients. A CT scan of the chest organs revealed lesions of 16% to 25% of the lungs as “frosted glass”, mainly in the peripheral parts of the lungs in 84% of patients, in 32%—an increase in mediastinal lymph nodes up to 16 mm. The general condition was mild in 68% of patients, and moderate in 32% of patients. All patients in this group were isolated, under medical supervision, and treated as outpatients. All were prescribed combined therapy: injections of Tamerit 200 mg 3 times daily, intramuscularly, 15 days; inhalation of Tamerit 100 mg 2 times daily, 15 days; interferon-alpha-2-beta 1 million units twice daily, 15 days. During the first week all patients (100%) had a significant reduction of cough, temperature to subfebrile, weakness, shortness, joint and muscle pains, smell and taste restored (68%). After two weeks of combined therapy, all clinical symptoms regressed completely, hematological parameters normalized. The control chest CT scan revealed no pathology in 68% of patients, in 32% of patients there was a reduction of infiltrative changes, a significant positive response to the ongoing therapy.

To determine the tactics of outpatient treatment, considering the clinical picture of the disease and the severity of the second group patients (with an uncomplicated course of new coronavirus infection), the choice was made in favor of an effective and safe combination: Tamerit—a medicine with immunomodulatory (endogenous interferon inducer), anti-inflammatory properties, which has its own antiviral effect against coronavirus infections and exogenous interferon-alfa-2-beta. None of the patients in the second group required hospitalization.
Group 3-15 patients diagnosed on the basis of clinical data, a CT chest scan and PCR diagnostics; the condition was moderate in 80% of patients and severe—in 20% of patients. Before the start of treatment we noted: fever up to 38-39° C. (100%), general weakness (100%), muscle and joint pains (100%), cough (more often dry)—in 80% of patients, scanty sputum with blood streaks—in 20% of patients, loss of smell and taste—in 86% of patients, headache—in 100%, pains in the stomach, loose stool—in 26% of patients, shortness of breath, shortness of breath, tightness and heaviness in the chest in 80% of patients, anxiety in 100% of patients, skin hemorrhagic rash in 6% of patients, in blood tests—expressed leukopenia in 100%, neutropenia in 100%, lymphocytosis in 100%, and later in 28% of patients—neutropenia, lymphopenia, monocytosis significant increase in ESR, interleukin-6, ferritin, and CRP levels. Positive test for COVID-19 in 100% of patients. On CT investigation of the chest organs: “ground glass” lung involvement 25-50%—in 54% of patients, 50-70%—in 40% of patients, one patient had 100% lung involvement. Radiological changes mainly in the peripheral parts of the lungs were revealed in 87% of patients, in 13%—in all lung fields and mediastinal lymph node enlargement up to 14-16 mm. 82% of patients with moderate severity pneumonia were isolated, were under medical observation on outpatient treatment. 18% of patients with history of chronic obstructive bronchitis, chronic pancreatitis were admitted to hospital in severe condition.

Three patients of the third group, in a serious condition, who were under inpatient treatment, in strict accordance with the “Temporary Methodological Recommendations “Prevention, Diagnosis and Treatment of New Coronavirus Infection (2019-nCoV)” (approved by The Russian Ministry of Health on Feb. 3, 2020, subsequent versions, including v.6 of 24 Apr. 2020) were prescribed complex therapy (antimalarial drug mefloquine 250 mg per day, azithromycin 500 mg per day, antithrombotic therapy with low-molecular heparins, interleukin-6 blocker kevzar). In 14% of patients, toxic drug-induced hepatitis manifestations (nausea, repeated vomiting, increased ALT, AST, CRP in blood tests) developed in response to the therapy. The expected clinical effect of the ongoing therapy was not obtained, correction of treatment was carried out and all 100% patients, including three inpatients, in order to prevent catastrophic development of the disease, toxic shock, DIC syndrome, “cytokine storm” were prescribed complex therapy: parenterally—Tamerit—a drug with immunomodulatory (inducer of endogenous interferon), anti-inflammatory properties, which has its own antiviral action against coronavirus infections; parenterally—broad spectrum antibacterial drugs—respiratory fluoroquinolones; rectally—exogenous interferon-alpha-2-beta; subcutaneously—antithrombotic therapy with low molecular weight heparin. Injections of Tamerit 200 mg 3 times a day, intramuscularly, 15 days; inhalations of Tamerit 150-200 mg 3 times a day, 15 days; interferon-alpha-2-beta 1 million units 2 times a day, 15 days; levofloxacin 500 mg once a day, oral, 15 days; fluconazole 150 mg on days 3, 7 and 15 of the course; enoxaparin 40 mg once a day, subcutaneously.

All patients in the third group received combination therapy:
Tamerit was prescribed 100-200 mg 3-4 times daily by intramuscular injection, depending on the severity of the condition until the relief of the main symptoms, then 1-2 times daily for 2 weeks, and then 100 mg by inhalation until complete recovery, in order to prevent possible pulmonary fibrosis and other complications;
Respiratory fluoroquinolone—levofloxacin 500 mg once daily, orally, for 2 weeks;
interferon-alpha-2-beta 1 million IU twice a day, 2 weeks;
antithrombotic therapy—enoxaparin 40 mg once a day, subcutaneously.

### EXAMPLE 4

### Use of Tamerit in a Patient with Severe Coronavirus Pneumonia (COVID-19+)

| Information | Complaints, | | | |
|---|---|---|---|---|
| about | clinical | | | |
| patient, | data on | | | |
| Diagnosis | admission | | | |
| COVID19 | and on | Results of instrumental | Laboratory | Drug |
| test | discharge | studies | findings | therapy |
| Patient G.N., | On admission: | 19.4.2020Γ.: Lung | 20.04.2020: Blood | From |
| 50 years old | complaints of: | MSCT revealed | tests: leukocytes- | 19.04.20: |
| stationary | worsening of | bilateral rounded | 3.32 × 10/⁹/l, | Mefloquine, |
| treatment | condition for | “frosted glass” type | oerythrocytes- | Kevzara, |
| from 19 Apr. to | three days in the | changes | 4.95 × 10/¹²/l, | Kaletra, |
| Aug. 5, 2020 | form of difficulty | (consolidation/reticular) | platelets- | Leflobakt, |
| Diagnosis: | in breathing, dry | located predominantly | 154 × 10/⁹/l, | Fluconazole, |
| U07.1 New | cough, increase | in the peripheral | neutrophils-48%, | Sulperazone, |
| coronavirus | in pronounced | anterior and posterior, | lymphocytes-33%, | Azithromycin, |
| infection | weakness and | upper and lower lung | ESR-22 mm/h. | Ceftriaxone, |
| SARS-Cov- | sweating, t-38.5- | regions. Fibrous bands +. | CRP-81.5 mg/L | Fraxiparin |
| 2(+) RNA | 39° C. | 44% lesions Total | (normal <5), | (cancellation |
| from | On admission, | score 11. CT-2. | ALT-21 units/l | on the 3rd day |
| Apr. 19, 2020. | the condition was | 24.4.2020Γ.: Lung | (norm <40), AST- | of treatment |
| B34.2 | moderately | MSCT revealed | 39 units/l (norm | due to |
| Community- | severe, and the | bilateral clear rounded | <40); | pronounced |
| acquired | pharynx was | “frosted glass” type | procalcitonin- | side effects- |
| bilateral | hyperemic. | changes | 0.141 ng/ml (less | nausea, |
| polysegmental | Breathing is | (consolidation/reticular) | than 0.5-low | vomiting). |
| pneumonia, | rigid, respiratory | located in the central | probability of | Since |
| severe course, | rate is 20 per | and peripheral anterior | sepsis), | 25.04.20: |
| DN-3. | minute. [symbol] | and posterior, upper | interleukin-6-26.5 | 1. Tamerit |
| Test for | [symbol] | and lower lung regions. | pg/ml (normal less | 200 mg v/m 3 |
| 19 Apr. 2020: | [symbol] | Fibrous bands +. 100% | than 7). Fibrinogen- | times a day; |
| 0000521784- | SpO₂ 99%, [symbol] | lesions Total score 25. | 5.0 g/l (1.69-3.92), | 2. Tamerit- |
| XF02 SARS- | 127/90 MM PT. CT. | CT-4. | prothrombin- | inhalation of |
| Cov-2 RNA | PS 98/ [symbol] ; | MSCT OGC | 116% (78-125). | 150 mg 2-3 |
| detected on | [symbol] | 08.05.2020: | 26.04.2020: | times a day. |
| Jul. 5, 2020: | [symbol] | There is a pronounced | Blood tests: | 3. Viferon |
| 2620- | [symbol] | positive dynamics to | leukocytes- | suppositories, |
| XF02 SARS- | [symbol] Stool | the ongoing treatment. | 4.07 × 10/⁹/l, | 1 mlned, 1 sv |
| Cov-2 RNA- | loosening. | Residual effects of | erythrocytes- | 2 times a day. |
| not detected. | Urination is | bilateral pneumonia. | 4.35 × 10/¹²/l, | |
| | normal. | CT-2. | thrombocytes- | |
| | | | 227 × 10/⁹/l, | |
| | | | neutrophils-70%, | |
| | | | lymphocytes- | |
| | | | 21%, ESR-22 | |
| | | | mm/h. ALT-46 | |
| | | | units/l (<40), AST- | |
| | | | 93 units/l (<40) | |
| | | | 28.04.2020Γ.: ALT- | |
| | | | 96 units/L (<40), | |
| | | | AST-126 units/L | |
| | | | (<40); CRP-335.5 | |
| | | | mg/L (<5), | |
| | | | interleukin-6- | |
| | | | 130.4 pg/mL, | |
| | | | ferritin->2000 | |
| | | | ng/ml (28-397); | |
| | | | procalcitonin- | |
| | | | 0.19 ng/mL. | |
| | | | 02.05.2020: blood | |
| | | | tests: leukocytes- | |
| | | | 3.87 × 10/⁹/l, | |
| | | | erythrocytes- | |
| | | | 4.32 × 10/¹²/l, | |
| | | | platelets- | |
| | | | 487 × 10/⁹/l, | |
| | | | neutrophils-68%, | |
| | | | lymphocytes- | |
| | | | 18%, monocytes- | |
| | | | 10%, eos-2%, | |
| | | | plasma cells-1%, | |
| | | | ESR-25 mm/h. | |
| | | | CRP-36.4mg/L | |
| | | | (<5), interleukin-6- | |
| | | | 924.6 pg/mL, | |
| | | | ferritin-973.3 | |
| | | | ng/ml (28-397). | |
| | | | 07.05.2020: CRP- | |
| | | | 3.6 mg/L (<5). | |

Patient G. N., 50 years old, on Apr. 19, 2020, after admission had a sharp increase of respiratory failure, clinical and laboratory manifestations of “cytokine storm” (leukopenia to 3.32×10/⁹/l, neutropenia—48% (normal 50-70), sharp increase of CRP to 81.5 mg/l (normal less than 5), interleukin-6 to 26.5 pg/ml (normal less than 7). The standard therapy (mefloquine, azithromycin, interleukin-6 blocker kevzar) started in accordance with the Provisional Recommendations of the Ministry of Health of the Russian Federation (6.0) caused on 21 Apr. 2020, on the third day of treatment the development of toxic drug-induced hepatitis (severe nausea, vomiting, in the biochemical blood tests of 26.04 and 28.05 the consecutive increase of ALT to 96 units/l (the norm is less than 40), AST to 126 units/l (the norm is less than 40), CRP to 335.5 mg/l, interleukin-6 to 130.6 pg/mL, ferritin over 2000 ng/mL (the norm is 28-397)). Correction of treatment was carried out—the above mentioned drugs were cancelled, antihistamine and hepatoprotective therapy were prescribed. By May 24, 2020 the patient's condition became grave: weakness, shortness of breath up to 24 min, oxygen saturation decreased to 86-88%, body temperature was at 38° C., neutrophilosis—70% (the norm is 50-70), lymphopenia—21% (the norm is 25-40) in the blood test. Since 24 Apr. 2020 the patient has been prescribed Tamerit 200 mg intramuscularly 3 times a day and Tamerit-inhalation 150 mg 2-3 times a day for two weeks, interferon-alpha-2-beta-suppositories, 1 mined, 1 suppository 2 times a day; Clexane 80 mg a day; Levofloxacin 500 mg a day. Changes in the therapy we were carrying out produced rapid positive dynamics of clinical and laboratory data: body temperature normalized, phenomena of respiratory insufficiency decreased, cough became less frequent, indices of systemic inflammation decreased. At the control MSCT of the chest organs on May 8, 2020, there was a pronounced positive dynamics on the conducted treatment. Residual effects of bilateral pneumonia. Due to a significant subjective improvement of his condition, the patient insisted on being discharged from the clinic.

Important features of clinical course of a new coronavirus infection SARS-Cov-2 RNA (COVID19) with development of pneumonia should be noted: at the beginning of the disease, radiological changes (according to MSCT of thoracic organs) precede by 5-7 days the appearance of complaints and deterioration of patient's condition. After the start of treatment, the picture is reversed: the complaints, clinical and laboratory manifestations regress, while the radiological positive dynamics is significantly, by 7-10 days, delayed. This peculiarity of the course of new SARS-Cov-2 RNA coronavirus infection (COVID19) requires prolonged therapy aimed at stopping clinical manifestations of systemic inflammation, prevention and treatment of possible complications (pulmonary fibrosis, vasculitis, etc.). It should also be noted that the identified feature of the course of the new coronavirus SARS-Cov-2 RNA infection (COVID19) is likely to lead to chronicity of systemic inflammation and, consequently, to subsequent early disability of patients who have had a new coronavirus SARS-Cov-2 RNA infection (COVID19).

### EXAMPLE 5

### Antiviral Activity of the Combination of Tamerit with Ribavirin in an Experimental SARS Infection Model

In a series of experiments, the efficacy of ribavirin in combination with Tamerit was studied in the SARS model (strain SARS-Covktp3) on male purebred white mice weighing 16-18 g obtained from Stolbovaya nursery. Ribavirin was administered orally and Tamerit was administered parenterally (subcutaneously) according to unified schedules (24 h before infection, 24 h, 48 h, 72 h, 96 h and 120 h after infection), infected with SARS-Cov virus at a dose of 10 LD₅₀. The animals were monitored for 14 days, recording the number of live and dead animals daily. The results are shown in Table 1.

As follows from the data presented, the antiviral ribavirin ensured immunity to SARS-CoV infection in infected animals depending on the dose used at the level of 10% to 50%, and the most effective of the ribavirin doses used was 500.0 mg/kg,

TABLE 1
Comparative evaluation of the protective efficacy of triazavirin in
combination with Tamerit and maktavirin in combination with
Tamerit in an experimental SARS model (SARS-Covktp3)

| | | | | Number of | | |
|---|---|---|---|---|---|---|
| | | | Contagious | animals | | |
| | | | dose of the | in the | | |
| | | | pathogen, | group, | Survival rate, | Protection, |
| Drug | Dose | | LD₅₀ | heads | % | % |
| Ribavirin | 100 | mg/kg | 10 | 10 | 10 (0 ÷ 44) | 10 (0 ÷ 44) |
| Ribavirin | 250 | mg/kg | 10 | 10 | 20 (2 ÷ 56) | 20 (2 ÷ 56) |
| Ribavirin | 500 | mg/kg | 10 | 10 | 50 (19 ÷ 81) | 50 (19 ÷ 81) |
| Ribavirin + | 100.0 | mg/kg | 10 | 10 | 50 (19 ÷ 81) | 50 (19 ÷ 81) |
| Tamerit | 0.20 | mg/ml | | | | |
| Ribavirin + | 250 | mg/kg | 10 | 10 | 100 (69 ÷ 100)* | 100 (69 ÷ 100) |
| Tamerit | 0.20 | mg/ml | | | | |
| Ribavirin + | 500.0 | mg/kg | 10 | 10 | 100 (69 ÷ 100)* | 100 (69 ÷ 100) |
| Tamerit | 0.20 | mg/ml | | | | |
| Tamerit | 0.20 | mg/ml | 10 | 10 | 50 (19 ÷ 81) | 50 (19 ÷ 81) |

| | | | | | |
|---|---|---|---|---|---|
| Control | — | 10 | 10 | 0 (0 ÷ 31) | — |

*Differences with control are significant at p < 0.05.

Tamerit itself, under the same conditions, protected infected animals from SARS virus infection at the level of 50% against the background of 100% lethality in the control. At the same time, the combined use of ribavirin with Tamerit significantly increased the level of antiviral protection and, depending on the used dose of ribavirin in combination, increased by 50-80% compared to the use without Tamerit alone. Thus, a clearly expressed ability of Tamerit to increase anti-infective activity when used in combination with the traditional antiviral agent ribavirin against SARS-CoV RNA virus has been revealed.

### EXAMPLE 6

### Antiviral Activity of Combination of Arbidol with Tamerit in Experimental MERS Infection Model

In a series of experiments on male purebred white mice weighing 16-18 g obtained from “Stolbovaya” cattery of RAS, the efficacy of Arbidol in combination with Tamerit was studied in the MERS model (strain MERS-CovN3/Jordanp3). The antiviral agents were administered orally, and Tamerit—parenterally (subcutaneously) according to the same schemes (24 h before infection, 24 h, 48 h, 72 h, 96 h and 120 h after infection) to MERS-CoV-infected patients at a dose of 10 LD₅₀. The animals were monitored for 14 days, recording the number of live and dead animals daily. The results are shown in Table 2.

As the data in Table 2 show, in the MERS model, the efficacy of the combined use of the antiviral drug with Tamerit was markedly higher than that registered in the case of using each of them alone. At the same time, the survival rate under the influence of Tamerit in combination increases by 40-50% compared to the use of antivirals alone.

TABLE 2
Comparative evaluation of the protective effectiveness of Arbidol
in combination with Tamerit on the experimental MERS model
(MERS-CovN3/Jordanp3 virus)

| | | | | Number | | |
|---|---|---|---|---|---|---|
| | | | | of | | |
| | | | Contagious | animals | | |
| | | | dose of the | in the | | |
| | | | pathogen, | group, | Survival rate, | Protection, |
| Drug | Dose | | LD₅₀ | heads | % | % |
| Arbidol | 60 | mg/kg | 10 | 10 | 10 (0 ÷ 44) | 10 (0 ÷ 44) |
| Arbidol | 135 | mg/kg | 10 | 10 | 60 (26 ÷ 83) | 60 (26 ÷ 83) |
| Arbidol + | 60 | mg/kg | 10 | 10 | 80 (44 ÷ 98)* | 80 (44 ÷ 98) |
| Tamerit | 0.2 | mg/ml | | | | |
| Arbidol + | 135, | mg/kg | 10 | 10 | 100 (69 ÷ 100)* | 100 (69 ÷ 100) |

| | | | | | |
|---|---|---|---|---|---|
| Tamerit | 0.2 | | | | |
| Tamerit | 0.2 | 10 | 10 | 50 (19 ÷ 81) | 40 (19 ÷ 81) |
| Control | — | 10 | 10 | 0 (0 ÷ 31) | — |

*Differences with control are significant at p<0.05.

As the data in Table 2 show, in the MERS model, the efficacy of the combined use of Arbidol with Tamerit was markedly higher than that registered in the case of use of each of them separately. At the same time, the survival rate under the influence of Tamerit in combination increases by 40-70% compared to the use of Arbidol alone.

The following are examples demonstrating the adjuvant properties of Tamerit when used as an adjuvant in the prevention of viral diseases. Example 7 demonstrates the efficacy of Tamerit as an adjuvant when used against Venezuelan equine encephalomyelitis. This example is a correct extrapolation for coronavirus because it not only refers to the prevention of viral disease, but the virus is a single-stranded RNA virus, like coronavirus.

### EXAMPLE 7

### Adjuvant Effect of Abidov's Adjuvant Against ILP Against Venezuelan Equine Encephalomyelitis

The evaluation of the adjuvant properties of Abidov's adjuvant in relation to the vaccine of Venezuelan equine encephalomyelitis (VVE) was carried out in “point” experiments, 10 mice weighing from 16 to 18 g were used at each point. Virus-containing material was administered to animals subcutaneously in a volume of 0.3 ml/mouse. The infectious doses of the virus were 2 and 10 of LD₅₀. The virus of Venezuelan equine encephalomyelitis (VEL), strain Trinidad was used for infection. The initial virus titer was 10⁷-10⁸ LD₅₀/ml. Cultural inactivated liquid VVE (VVE) was used for immunization (ser. 145; control No. 1244). The adjuvant was used at a dose of 150 μg/individual, administered intramuscularly according to various schemes. IMP was used once, administered intramuscularly in a volume of 0.5 ml, immunizing dose of IMP was 0.1 of human dose.

As follows from the presented data, VVE ensured the survival rate of 15% (10 LD₅₀) and 70% (2 LD₅₀) of infected animals administered once at a dose equal to 0.1 of human dose 21 days before infection, depending on the infecting dose of the virus.

The results of conducted studies are presented in Table 2.

TABLE 2
Assessment of the adjuvant activity of Abidov's adjuvant against IMP
against Venezuelan equine encephalomyelitis

| | | Infectious | |
|---|---|---|---|
| | | dose of the | Survival rate, |
| Scheme of Abidov's | | pathogen | M (M − tm- |
| adjuvant administration in | Method of adjuvant | (LD₅₀ | M + tm), |
| relation to VVE and infection | administration | amount) | % |
| Simultaneously with VVE in | Intramuscularly | 10.0 | 15 (2-45) |
| one syringe 21 days before | | | |
| infection | | 2.0 | 40 (12-74) |
| Simultaneously with VVE in | Intramuscularly | 10.0 | 40 (12-74) |
| different syringes 21 days | | | |
| before infection | | 2.0 | 100 (69-100)* |
| Separately from VVE 3, 2, 1 | Intramuscularly | 10.0 | 40 (12-74) |
| days before infection | | 2.0 | 100 (69-100)* |
| Without VVE 21 days | Intramuscularly | 10.0 | 40 (12-74) |
| before infection | | 2.0 | 70 (35-93)* |
| Without VVE 3, 2, 1 days | Intramuscularly | 10.0 | 60 (26-83) |
| before infection | | 2.0 | 100 (69-100)* |
| Not administered (control 1) | — | 10.0 | 15 (2-45) |
| | | 2.0 | 70 (35-93)* |
| Not administered (control 2) | — | 10.0 | 0 (0-31) |
| | | 2.0 | 15 (2-45) |

Control 1-animals were immunized only with VVE;
Control 2-VVE and Abidov's adjuvant were not administered;
*differences with indicators in control groups are considered to be significant at P < 0.05

Administration of only Abidov's adjuvant in the same period ensured the survival rate of 40% (10 LD₅₀) and 70% (2 LD₅₀) of mice infected with the VVE virus. The use of Abidov's adjuvant on a multiple scheme was more effective. At the same time, the survival rate of infected mice was 60-100% depending on the infecting dose of the virus.

With simultaneous administration of VVE and Abidov's adjuvant in one syringe, the survival rates of infected animals ranged from 15 to 40% and were either at the level or slightly lower than in groups of animals that were administered with only VVE or only adjuvant. The simultaneous use of VVE and adjuvant in different syringes was more effective. In this case, the survival rate of infected animals was 40% and 100% depending on the size of the infecting dose of the VVE virus, when infected with the virus at a dose of 10 LD₅₀₁ and 2 LD₅₀ respectively, which turned out to be 25-30% higher than the similar indicator registered in groups of animals, immunized only with VVE.

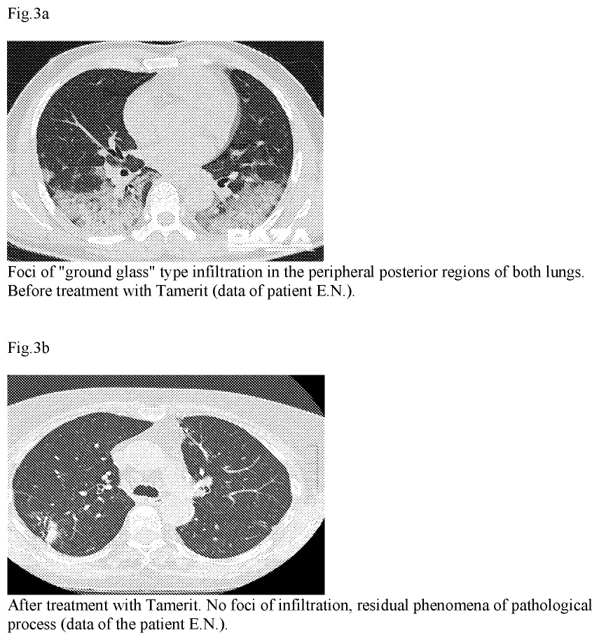

According to the results of the research, it is shown that:
the use of “Tamerit” in the complex therapy of patients with COVID-19 improved the condition of patients and reduced the duration of clinical manifestations of the acute phase of the disease by about 1.5-2 weeks of days compared to the use of therapy that includes only antimalarials, antibacterials and interferon drugs;
none of the observed patients treated with Tamerit had indications for artificial lung ventilation;
according to the results of computed tomography, the development of early pulmonary fibrosis was not observed in any patient (FIG. 3a, 3b);

The totality of the data presented allows us to consider the drug “Tamerit” as a highly effective nonspecific means of anti-infective protection, it is advisable for inclusion in the scheme of prevention, integrated treatment of antiviral, such as coronavirus infection, including at the recovery stage of treatment, involving the use in addition to Tamerit drugs interferon, azoloazine series, drugs with antimalarials, antibiotics and other combinations, in the treatment of other associated nosologies with the use of drugs, such as antiparasitic, antifungal, anthelminthic activity, etc. Due to its immunomodulatory activity, Tamerit enhances the body's defenses, especially the nonspecific immunological resistance, thereby allowing traditional antiviral agents to show more active therapeutic properties and reduce the acute period of the disease by about 1.5-2 weeks.

The above examples unambiguously testify to the industrial applicability of the claimed invention, as they show both the reproducibility and the realization of the purpose. At the same time, they reliably confirm the validity of the breadth of the claimed claims, as according to the national legislation to justify the validity of the degree of generalization used by the applicant in disclosing the essential feature of the invention, information on private forms of realization of this essential feature is provided. Sufficient examples of the invention are presented above, confirming the possibility of obtaining the technical result indicated by the applicant by using private forms of realization of the essential feature of the invention.

### LIST OF REFERENCES

1. Vijay R, Perlman S. ScienceDirect Middle East respiratory syndrome and severe acute respiratory syndrome. Curr. Opin. Virol. 2016; 16:70-76.
2. Peck K M, Burch C L, Heise M T, Baric R S. Coronavirus host range expansion and Middle East respiratory syndrome coronavirus emergence: biochemical mechanisms and evolutionary perspectives. Annu. Rev. Virol. 2015; 2(1):95-117.
3. Banik G R, Khandaker G, Rashid H. Middle East respiratory syndrome coronavirus ‘MERS-CoV’: current knowledge gaps. Paediatr. Respir. Rev. 2015; 16(3):197-202.
4. Chan J F W, Lau S K P, To K K W, Cheng V C C, Woo P C Y, Yuen K-Y. Middle East respiratory syndrome coronavirus: another zoonotic betacoronavirus causing SARS-like disease. Clin. Microbiol. Rev. 2015; 28(2):465-522.
5. Hilgenfeld R, Peiris M. From SARS to MERS: 10 years of research on highly pathogenic human coronaviruses. Antiviral. Res. 2013; 100(1):286-295.
6. Drosten C, Günther S, Preiser W, et al. Identification of a novel coronavirus in patients with severe acute respiratory syndrome. N. Engl. J. Med. 2003; 348(20):1967-1976.
7. Rota P A, Oberste M S, Monroe S S, et al. Characterization of a novel coronavirus associated with severe acute respiratory syndrome. Science. 2003; 300(5624):1394-1399.
8. Kuiken T, Fouchier R A M, Schutten M, et al. Newly discovered coronavirus as the primary cause of severe acute respiratory syndrome. Lancet. 2003; 362(9380):263-270.
9. Drosten C, Preiser W, Günther S, Schmitz H, Doerr H W. Severe acute respiratory syndrome: identification of the etiological agent. Trends. Mol. Med. 2003; 9(8):325-327.
10. Alsahafi A J, Cheng A C. The epidemiology of Middle East respiratory syndrome coronavirus in the Kingdom of Saudi Arabia, 2012-2015. Int. J. Infect. Dis. 2016; 45:1-4.
11. Drexler J F, Corman V M, Drosten C. Ecology evolution and classification of bat coronaviruses in the aftermath of SARS. Antiviral. Res. 2014; 101(C):45-56.
12. Milne-Price S, Miazgowicz K L, Munster V J. The emergence of the Middle East respiratory syndrome coronavirus. Pathog. Dis. 2014; 71(2):121-136.
13. Weber D J, Rutala W A, Fischer W A, Kanamori H, Sickbert-Bennett E E. Emerging infectious diseases: focus on infection control issues for novel coronaviruses (severe acute respiratory syndrome-CoV and Middle East respiratory syndrome-CoV), hemorrhagic fever viruses (Lassa and Ebola), and highly pathogenic avian influenza viruses, A(H5N1) and A(H7N9) Am. J. Infect. Control. 2016; 44(Suppl. 5):e91-e100.
14. Drosten C. Is MERS another SARS? Lancet Infect. Dis. 2013; 13(9):727-728.
15. Berger A, Drosten C, Doerr H W, Stürmer M, Preiser W. Severe acute respiratory syndrome (SARS)—paradigm of an emerging viral infection. J. Clin. Clin. Virol. 2004; 29(1):13-22.
16. Yang X-L, Hu B, Wang B, et al. Isolation and characterization of a novel bat coronavirus closely related to the direct progenitor of severe acute respiratory syndrome coronavirus. J. Virol. 2016; 90(6):3253-3256.
17. Guan Y, Drosten C. ScienceDirect editorial overview: emerging viruses: interspecies transmission. Curr. Opin. Virol. 2016; 16:v-vi.
18. Yount B L, Debbink K, Agnihothram S, et al. A SARS-like cluster of circulating bat coronaviruses shows potential for human emergence. Nat. Med. 2015; 21(12):1508-1513.
19. Pfefferle S, Oppong S, Drexler J F, et al. Distant relatives of severe acute respiratory syndrome coronavirus and close relatives of human coronavirus 229E in bats, Ghana. Emerging Infect. Dis. 2009; 15(9):1377-1384.
20. Corman V M, Kallies R, Philipps H, et al. Characterization of a novel betacoronavirus related to Middle East respiratory syndrome coronavirus in European hedgehogs. J. Virol. 2014; 88(1):717-724.
21. Yadam S, Bihler E, Balaan M. Acute respiratory distress syndrome. Crit. Care. Nurs. Q. 2016; 39(2):190-195.
22. Sweeney R M, McAuley D F. Acute respiratory distress syndrome. Lancet. 2016 Epub ahead of print.
23. Mackay A, Al-Haddad M. Acute lung injury and acute respiratory distress syndrome. Contin. Educ. Anaesth. Crit. Care. Pain. 2009; 9(5):152-156.
24. Medzhitov R, Schneider D S, Soares M P. Disease tolerance as a defense strategy. Science. 2012; 335(6071):936-941.
25. Ayres J S, Schneider D S. Tolerance of infections. Annu. Rev. Immunol. 2012; 30:271-294.
26. Herold S, Mayer K, Lohmeyer J. Acute lung injury: how macrophages orchestrate resolution of inflammation and tissue repair. Front. Immunol. 2011; 2:65.
27. Darrell Ricke, Robert W. Malone. Medical Countermeasures Analysis of 2019-nCoV and Vaccine Risks for Antibody-Dependent Enhancement (ADE)//SSRN Electronic Journal.-2020.-ISSN1556-5068.-doi:10.2139/ssm.3546070.
28. Vatutin N. T., Eschenko E. B. Lymphopenia: main causes of development//Archives of Internal Medicine.-2016.- [symbol] 2(28).-C. 22-27.
29. Jiang Gu, Clive R. Taylor. Acute Immunodeficiency, Multiple Organ Injury, and the Pathogenesis of SARS//Applied Immunohistochemistry & Molecular Morphology.-2003 December.-C. 281-282.-ISSN 1541-2016.-doi:10.1097/00129039-200312000-00001.
30. Jiang Gu, Clive R. Taylor. Acute Immunodeficiency, Multiple Organ Injury, and the Pathogenesis of SARS//Applied Immunohistochemistry & Molecular Morphology.-2003 December.-C. 281-282.-ISSN 1541-2016.-doi:10.1097/00129039-200312000-00001.
31. British Patent No. 0361831, published 4 Apr. 1990, MKI⁴ A61K 31/70.
32. UK Patent No 0366287, published 2 May 1990, MKI⁴ A61K 31/70.
33. Patent of Japan, No. 1-55250, 22 Nov. 1989, MKI⁴ A61K 31/70/c07H.
34. Patent of France, No. 2-12933, 30 Mar. 1990, A61K 37/66.
35. “Study of the combined effect of remantadine and ribavirin on experimental influenza infection”//Question. Virosol.-1981.-No. 6.-C.697-701.
36. Study of combined effect of rimantadine and ribavirin on reproduction of Sindbis virus in cell culture//Question. Virosol.-1980.- [symbol] 5.-C.608-611.
37. Combined use of antiviral drugs//Question. Virosol.-1984.-No. 6.-C.644-656.
38. Abidov M. T. Toxic syndrome, pathogenesis and methods of correction. Materials for the doctoral dissertation. 1994 Γ.
39. Bulletin of Experimental Biology and Medicine, ed.
40. Report by N. Filatov at the meeting with V. Putin on the sanitary-epidemiological situation in the Russian Federation, 21 Apr. 2020, https://www.vshouz.ru/news/pravitelstvo/10058/

1. A vaccine for protection against coronavirus infection, characterized in that it contains as an adjuvant the drug Tamerit in an effective amount, as well as pharmaceutically acceptable carriers and/or diluents.

2. A method of protection against coronavirus infection, characterized by the use of drugs containing as an active substance salts of aminophthalhydrazide derivatives in the form of dihydrate, monohydrate, anhydrate, in any crystal form, including the drug Tamerit, in a dose from 0.01 to 4000 mg/kg to a subject in need.

3. Method of protection against coronavirus infection, characterized by the fact that to increase the clinical and laboratory efficacy achieved by antiviral agents azoloazinovyh (Triazavirin®, Maktavirin®), or antimalarials, or preparations of interferon, preparations containing as an active substance salts of aminophthalhydrazide derivatives in the form of dihydrate, monohydrate, anhydrate, in any crystalline form, including Tamerit, at a dose of 0.01 to 4000 mg/kg to the sub-unit in need are used in combination with the above preparations.
